(19) United States
(12) Patent Application Publication
(10) Pub. No.: US 2005/0090474 A1
(43) Pub. Date: Apr. 28, 2005

(54) Methods and compositions for enhancing and inhibiting fertilization

(76) Inventor: Zvi Naor, Tel Aviv (IL)

Correspondence Address: Martin Moynihan;Anthony Castorina, 2001 Jefferson Davis Highway, Suite 207, Arlington, VA, 22202, US

(21) Appl. No.: 10/498,830

(22) PCT Filed: Jan. 16, 2003
(86) PCT No.: PCT/IL03/00044

(30) Foreign Application Priority Data
Jan. 16, 2002 (US) 60348379

(51) Int. Cl. A61K 31/56

(52) U.S. Cl. 514/170; 514/2

(57) ABSTRACT

A method of contraception is provided. The method comprises providing to a subject an amount of a p38 activator and/or an ERK inhibitor capable of substantially reducing sperm motility. Also provided is a method of enhancing fertility comprising providing to a subject a therapeutically effective amount of a p38 inhibitor and/or an ERK activator, thereby enhancing fertility.

### FIELD AND BACKGROUND OF THE INVENTION

The present invention relates to methods and compositions suitable for fertilization and contraception and, more particularly, to the use of MAPK activators or inhibitors in pharmaceutical compositions suitable for contraception and enhancing fertilization in mammals.

In the modern world, family planning is a major issue for most couples in terms of providing education and financial support to their children. In the underdeveloped world, on the other hand, the population is constantly increasing due to unprotected sexual intercourses and lack of family planning awareness. Therefore, there is a need for convenient contraceptive methods useful for family planning which are applicable for both males and females.

Female Contraception Methods

Female contraception methods are mainly based on the inhibition of ovulation. High levels of the progesterone and estrogen hormones consumed as contraceptive drugs negatively regulate the secretion of the luteinizing hormone-releasing hormone (LHRH) from the hypothalamus which results in a decreased production of the luteinizing hormone (LH) and follicle stimulating hormone (FSH) from the pituitary. Low blood levels of LH and FSH inhibit the normal cyclic rise in LH (i.e., LH surge), which triggers the growth, maturation and rupture of the Graafian follicle during ovulation.

Several hormone-related methods are currently available for female contraception. These include oral contraception including a combination of estrogens and progesterones or progesterone alone, long acting intramuscular injections of progesterones (e.g., medroxyprogesterone acetate in DepoProvera™), subdermally released progestin (Norplant™) and intrauteral hormonal releasing systems (e.g., Mirena™). Other methods are based on agonists or antagonists of progesterone (e.g., tetrahydronaphthofuranone or tetrahydrobenzindolone derivatives), LHRH, gonadotropins (e.g., Danazol™), or anti human Chorionic Gonadotropin (hCG) antibodies (as described in U.S. Pat. No.: 6,469,139).

The non-hormonal related methods include barriers (e.g., a diaphragm with a flexible cervical dome), spermicides (as described in WO patent NO: WO0072839A1), intrauterine devices (IUD) or sterilization by tube ligation.

However, the use of current female contraceptive methods is often accompanied by undesirable side effects.

Hormone-based contraceptive drugs may cause headaches, mood changes, nervousness, abdominal cramps, dizziness, weakness or fatigue, nausea, vaginal irritation, breast swelling and tenderness, bloating, swelling of the hands or feet, backache, depression, mastalgia, insomnia, acne rashes, hot flashes and joint pains and vomiting. In addition contraceptive hormones slightly increase the risk of a cardiovascular disease, especially in subjects having a family history of early coronary disease, heavy smoking subjects and subjects suffering from hypercholesterolemia, hypertension and diabetes. Short term and long term—acting progesterone analogues have been causally implicated in various disorders such as deep vein thrombosis, pulmonary embolus, breast cancer, cervical cancer, pseudotumor cerebri, stroke, hypertension and thrombocytopenia, [Wysowski and Green (1995), Obstet. Gynecol. 85: 538-42].

Nonhormonal related contraception methods are basically inefficient (e.g., the use of a diaphragm might result in up to 14% unwanted pregnancies) and associated with physical discomfort. On the other hand the use of an UD is often associated with side effects such as irregular vaginal bleeding.

Male Contraception Methods

Hormonal related compositions are also used as male contraceptives, such as the inhibition of spermatogenesis by regulating the levels of FSH and androgens. Contraceptive formulations, such as Progestin or ester testosterone undecanoate in combination with norethisterone enanthate (Kamischke et al., 2002) suppress testicular spermatogenesis while feedback regulating LH and FSH from the hypothalamus and the pituitary glands.

However, these hormonal contraceptive methods have several side effects such as increase in body weight, erythrocytes, hemoglobin, and hematocrit and decrease in high-density lipoprotein cholesterol and alkaline phosphatase (Kamischke et al., 2002).

Other male contraceptive methods include the use of disposable condoms or sterilization by vasectomy.

However, contraception methods may account for only a subset of the population in the modern world. For many couples, spontaneous pregnancies may never occur since at least one spouse suffering from a medical condition interfering with his/her fertility potential.

Infertility

About 15% of all couples in the modern world have fertility problems that require medical interventions. Among them, 20% show female hormonal defect, 30% have female peritoneal factor, and 40% show a male factor etiology [The Merck Manual, MSD, 15^{th} Edition, 1987]. Therefore, there is also a need for useful methods and/or compositions capable of enhancing fertility.

Female Infertility Treatments

Most female infertility treatments are directed towards the induction of ovulation. The most commonly used fertility drug is the nonsteroidal antiestrogen clomiphene which binds to estrogen receptors in the hypothalamus and the pituitary and blocks the negative feedback exerted by ovarian estrogens. Other fertility drugs include commercial preparations of the human gonadotropins (e.g. FSH, LH or HCG), which stimulate folliculogenesis and steroidogenesis.

Male Fertility Treatments

Male infertility can result from hypogonadotrophic hypogonadism, retrograde ejaculation, disturbed hormonal regulation and deficiency in sperm motility. In many cases fertility is dependent on the number and quality of the sperms present during ejaculation. In a normal human semen sample there are approximately 4−40×10⁷ sperms, which upon ejaculation swim actively forward, cross through the uterine cervix to the opening of the fallopian tube, wherein about 250 sperm reach the fertilization site by chemotaxis (FIG. 1). In the uterus, sperm become capacitated (i.e., capable to fertilize), acquire the ability to undergo the acrosome reaction (AR) and become hyperactivated. This hyperactivation is required for the sperm to penetrate the egg's extracellular matrix (zona pellucida). When sperm reach the isthmus in the oviduct female reproductive tract they slow down and resume their hyperactivated motility only after ovulation, probably due to the rise in progesterone and other yet undefined chemical attractants [FIG. 2 and Eisenbach and Tur-Kaspa, 1999; Garbers, Nature 413: 579, 2001). The various cycles and forms of sperm motility occurring inside the female reproductive tract suggest that biochemical processes regulate each phase of the spermatozoa life cycle and that male fertility is partially dependent on factors present in the female genital tract.

Male fertility drugs targeted towards hormonal regulation, such as antiestrogens, androgens, aromatase-inhibitors, mast cell blockers, zinc and pentoxifylline, have been shown to have inconsistent results in enhancing male fertility (Haidl, 2002, Drugs, 62: 1741-53].

There is thus a widely recognized need for, and it would be highly advantageous to have methods and compositions suitable for contraception and enhancing fertility devoid of the above limitations.

### SUMMARY OF THE INVENTION

According to one aspect of the present invention there is provided a method of contraception comprising providing to a subject an amount of a p38 activator and/or an ERK inhibitor capable of substantially reducing sperm motility.

According to another aspect of the present invention there is provided a use of an ERK inhibitor and/or a p38 activator for the manufacture of a medicament useful as a contraceptive

According to yet another aspect of the present invention there is provided an article-of-manufacture comprising packaging material and a pharmaceutical composition identified as a contraceptive being contained within the packaging material, the pharmaceutical composition including, as an active ingredient, an ERK inhibitor and/or a p38 activator and a pharmaceutically acceptable carrier.

According to further features in preferred embodiments of the invention described below, the subject is a female and the step of providing is effected via intravaginal administration of the p38 activator and/or ERK inhibitor.

According to still further features in the described preferred embodiments the subject is a male and the step of providing is effected via genital administration of the p38 activator and/or ERK inhibitor.

According to still further features in the described preferred embodiments the p38 activator is selected from the group consisting of p38 activating growth factor, MKK, Rac, Cdc42 and PAK1.

According to still further features in the described preferred embodiments the p38 activating growth factor is selected from the group consisting of IL-1, IL-I-receptor, TNF, LPS, TRAF6 and TAB1/2.

According to still further features in the described preferred embodiments the MKK is selected from the group consisting of MKK3, MKK4 and MKK6.

According to still further features in the described preferred embodiments the Rac is selected from the group consisting of Rac(V12) and Rac(L61).

According to still further features in the described preferred embodiments the Cdc42 is selected from the group consisting of Cdc42(Q61L) and Cdc42(V12).

According to still further features in the described preferred embodiments the p38 activator is a condition selected from the group consisting of physical stress, chemical stress and osmotic shock.

According to still further features in the described preferred embodiments the ERK inhibitor is selected from the group consisting of a PKC inhibitor, a Ras inhibitor, a dominant negative Ras, a Raf-1 inhibitor, a dominant negative Raf-1, a MEK inhibitor, a dominant negative MEK, a dominant negative ERK and an ERK inhibitor.

According to still further features in the described preferred embodiments the PKC inhibitor is selected from the group consisting of GF109203X, PKC 19-31, PKC 19-36, staurosporine and G06976.

According to still further features in the described preferred embodiments the Ras inhibitor is a Ras inhibitory peptide.

According to still further features in the described preferred embodiments the dominant negative Ras is selected from the group consisting of Ras(S17N) and Ras(S17W).

According to still further features in the described preferred embodiments the Raf-1 inhibitor is selected from the group consisting of Raf-1 kinase inhibitor I (5-Iodo-3[(3,5-dibromo-4 hydroxyphenyl) methylene]-2 indolinone) and ZM 336372.

According to still further features in the described preferred embodiments the dominant negative Raf-l is selected from the group consisting of Raf-1(K375W), Raf(C4B), Raf 301 and Raf(S621A).

According to still further features in the described preferred embodiments the MEK inhibitor is selected from the group consisting of PD98059, U0126 and U0125.

According to still further features in the described preferred embodiments the dominant negative MEK is selected from the group consisting of MEK1(K97A), MEK1(K97M), MEK2(K101A) and MEK1/2(KAMEK).

According to still further features in the described preferred embodiments the dominant negative ERK is selected from the group consisting of ERK1(K71R) and ERK2(K52R).

According to still further features in the described preferred embodiments the ERK inhibitor is selected from the group consisting of PD98059, PD184352, U0126, ITU, Ste-MEK1₁₃ and MTP_{TAT}-G-MEK1₁₃.

According to still further features in the described preferred embodiments the amount of p38 activator and/or ERK inhibitor does not exceed 10 mg/kg body weight.

According to still another aspect of the present invention there is provided a method of enhancing fertility comprising providing to a subject a therapeutically effective amount of a p38 inhibitor and/or an ERK activator, thereby enhancing fertility.

According to an additional aspect of the present invention there is provided a use of an ERK activator and/or a p38 inhibitor for the manufacture of a medicament for enhancing fertility.

According to yet an additional aspect of the present invention there is provided an article-of-manufacture comprising packaging material and a pharmaceutical composition identified for enhancing fertility being contained within the packaging material, the pharmaceutical composition including, as an active ingredient, an ERK activator and/or a p38 inhibitor and a pharmaceutically acceptable carrier.

According to still an additional aspect of the present invention there is provided a method of enhancing sperm motility comprising:(a) obtaining a sperm cell sample; and (b) contacting the sperm cell sample with an ERK activator and/or p38 inhibitor, thereby enhancing sperm motility.

According to still further features in the described preferred embodiments the method further comprising: (c) isolating sperm cells exhibiting enhanced motility from the sperm cell sample.

According to still further features in the described preferred embodiments the subject is a female and the step of providing is effected via intravaginal administration of the p38 inhibitor and/or ERK activator.

According to still further features in the described preferred embodiments the subject is a male and the step of providing is effected via genital administration of the p38 inhibitor and/or ERK activator.

According to still further features in the described preferred embodiments the method further comprising providing to the subject a therapeutically effective amount of progesterone.

According to still further features in the described preferred embodiments the p38 inhibitor is selected from the group consisting of Rap, a dominant negative p38, a dominant negative Rac, a dominant negative Cdc42, a dominant negative MKK, a dominant negative Ras, a dominant negative PAK1 and a p38 inhibitor.

According to still further features in the described preferred embodiments the dominant negative p38 is p38betaAGF.

According to still further features in the described preferred embodiments the dominant negative Rac is Rac(T17N).

According to still further features in the described preferred embodiments the dominant negative Cdc42 is Cdc42(T17N).

According to still further features in the described preferred embodiments the dominant negative MKK is selected from the group consisting of MKK3(T193A), MKK4(S220A) and a dominant negative MKK6.

According to still further features in the described preferred embodiments the dominant negative Ras is selected from the group consisting of Ras(S17N) and Ras(S17W).

According to still further features in the described preferred embodiments the dominant negative PAK1 is PAK1(K299R).

According to still further features in the described preferred embodiments the p38 inhibitor is selected from the group consisting of 2-(4-Chlorophenyl)-4-(4-fluorophenyl)-5-pyridin-4-yl-1,2-dihydropyrazol-3-one, SC68376, SB203580(Iodo), SB202190, SB203580, SB203580(Sulfone), PD169316, SB220025, SKF-86002, SB239063, ML 3163 and a thienyl urea analog (C₁₇H₂ON₂O₃S).

According to still further features in the described preferred embodiments the ERK activator is selected from the group consisting of Ras, Raf-1, MEK1/2, a PKC activator and UV induction.

According to still further features in the described preferred embodiments the Ras is selected from the group consisting of Ras(G12V) and Ras(L61).

According to still further features in the described preferred embodiments the Raf-1 is Raf(BXB).

According to still further features in the described preferred embodiments the MEK1/2 is ΔN-EEMEK.

According to still further features in the described preferred embodiments the PKC activator is selected from the group consisting of TPA, diC8, OAG and arachidonic acid.

According to still further features in the described preferred embodiments the amount of p38 inhibitor and/or ERK activator does not exceed 10 mg/kg body weight.

According to yet a further aspect of the present invention there is provided a method of determining quality of a semen sample, the method comprising determining p38 activity in sperm cells of the semen sample, the p38 activity being inversely indicative of sperm cell motility, thereby determining the quality of the semen sample.

According to still further features in the described preferred embodiments determining p38 activity is effected by employing an antiphosphorylated p38 antibody.

According to still further features in the described preferred embodiments determining p38 activity is effected by a kinase activity assay is an in-gel kinase assay.

According to still a further aspect of the present invention there is provided a kit for determining quality of a semen sample, the kit comprising a container including a reagent suitable for determining p38 activity in sperm cells of the semen sample.

According to still further features in the described preferred embodiments the reagent is an antiphosphorylated p38 antibody.

According to still further features in the described preferred embodiments the kit further comprising a support for attaching the sperm cells.

According to still further features in the described preferred embodiments the kit further comprising reagents suitable for p38 detection.

The present invention successfully addresses the shortcomings of the presently known configurations by providing methods and compositions for enhancing and inhibiting fertilization.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are schematic illustrations describing the path of human spermatozoa in the female genital tract from insemination to fertilization, adopted from Eisenbach and Tur-kaspa (1999). FIG. 1a illustrates the distribution of sperm in woman genital tract subsequent to insemination. FIG. 1b illustrates a proposed sequence of events in the mammalian female genital tract. The yellow curved bar indicates the region in which turnover of capacitated spermatozoa may occur. The egg and the cumulus oophorus surrounding it (together seen as a yellow circle in the ampulla) are enlarged to make the egg-cumulus visible.

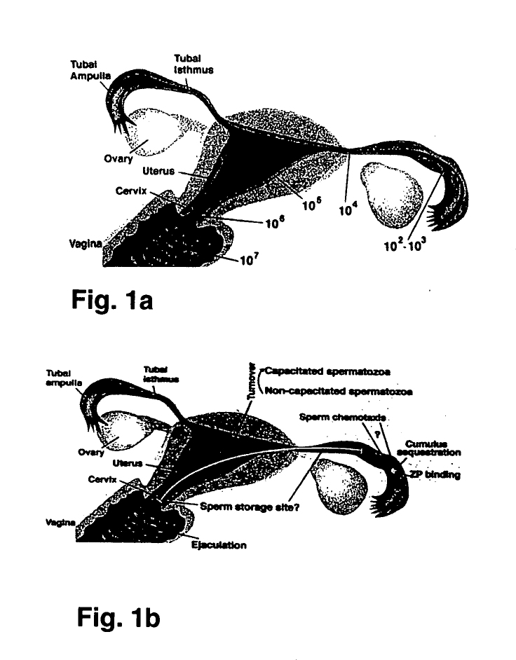

FIG. 2 is a schematic illustration describing the changes in sperm motility from production to fertilization as adapted from Garbers [Nature 413: 579, 2001].

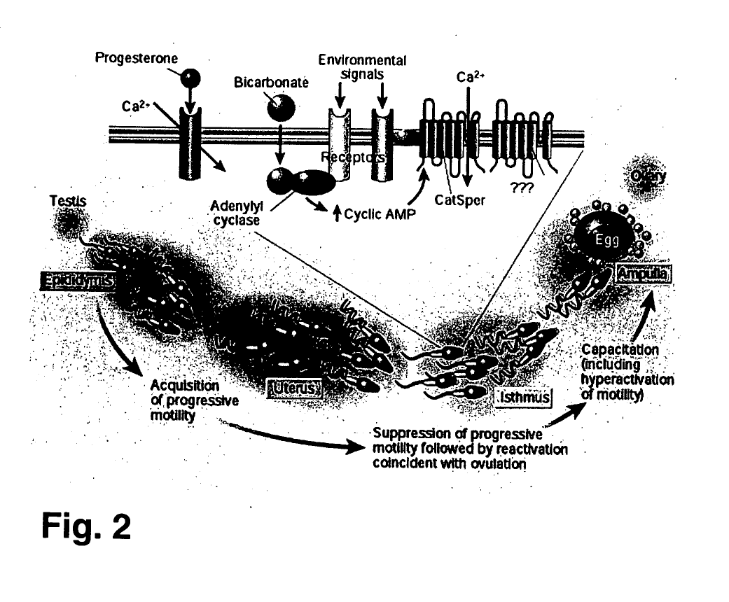

FIG. 3 is a schematic illustration of the mitogen activated protein kinase (MAPK) cascades as adapted from Seger and Krebs, (1995).

FIGS. 4a-f are autoradiograms depicting expression and activity of MAPK proteins in human spermatozoa. LβT2 cells were treated for 60 min with 50 ng/ml 12-O-Tetradecanoylphorbol-13-acetate (TPA, FIGS. 4a-f, lane 1) and capacitated human spermatozoa were treated for 60 min with 0.2 M vanadate (FIGS. 4a-f, lane 3) or were remained untreated (FIGS. 4a-f, lane 2). Cell lysates were subsequently prepared and subjected to Western blot analysis with antibodies directed at the active, phosphorylated form of ERK, p38 and JNK (FIGS. 4a, 4c and 4e, respectively), or with antibodies directed at the unphosphorylated form of ERK, p38 and JNK (FIGS. 4b, 4d and 4f, respectively). Note the increased band intensity observed with the antibody against activated-ERK in vanadate-treated spermatozoa (FIG. 4a, lane 3) as compared with the weak signal observed in untreated human spermatozoa (FIG. 4a, lane 2).

FIGS. 5a-c are bright field images illustrating the intra sperm localization of ERK cascade proteins in human spermatozoa using ERK cascade specific antibodies. Note the specific immuno-staining in the sperm neck and in patches along the entire tail using antibodies against ERK (FIG. 5a), Raf 1 (FIG. 5c), MEK (FIG. 5d) and m-SOS (not shown) and the disappearance of the specific signal following pre-absorption of the primary antibody with the ERK antigen (FIG. 5b). Tubulin immuno-staining revealed a specific and continuous labeling along the tail but no staining in the sperm neck (FIG. 5e). Magnification is X 1500.

FIGS. 6a-c are electron microscopy images illustrating a high-power localization of ERK in the human spermatozoa using a polyclonal anti ERK antibody and a secondary goat anti rabbit gold-labeled (8 nm particles) antibody. ERK gold-immuno-staining could be seen in segmented column of the sperm neck and the outer dense fibers but not in the mitochondria (FIG. 6a, magnification X 4750). ERK labeling was also observed in the outer dense fibers and the axoneme of the mid and principal pieces of the sperm (FIG. 6b, magnification X 4750). No ERK labeling was observed in the mitochondria and fibrous sheath. ERK labeling of the outer dense fibers and the axoneme was also observed in a cross section of the principle piece (FIG. 6c, magnification X 13,000).

FIGS. 7a-b are bright field images illustrating the intra sperm localization of p38 using an anti p38 antibody. Note the specific immunostaining in the sperm neck and in patches throughout the tail (FIG. 7a) and the disappearance of the specific signal following pre-absorption of the antibody with p38 antigen (FIG. 7b).

FIG. 8 is a graphic representation of the effect of progesterone and MAPK inhibitors on human spermatozoa motility. Human spermatozoa (5×10⁷/ml) were incubated with PD98059 (ERK inhibitor, at 50 μM, FIG. 8, marked with “PD”) and SB203580 (p38 inhibitor, at 50 μM, FIG. 8, marked with “SB”), in the presence or absence of progesterone (at 5 μg/ml, FIG. 8, marked with “P”) for the indicated time. Percent motile spermatozoa was determined in aliquots of 10 μl. Results are expressed as mean ±SEM of three experiments. Note the significant increase in sperm motility in the presence of the p38 inhibitor (FIG. 8, marked with “SB”) and the normal motility observed in the presence progesterone and p38 inhibitor.

### DESCRIPTION OF THE PREFERRED EMBODIMENTS

The present invention is of compositions and methods which can be used to enhance or inhibit fertilization.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Poor function of spermatozoa accounts for twenty five percent of all human infertility. In vitro fertilization (IVF) is one of the only effective treatments for such infertility problems [Winston and Handyside (1993) Science 260: 932-6], however success rates of IVF are limited since poor sperm function does not allow enough sperm to contact and penetrate the egg. Most previous strategies to overcome the difficulties of IVF have involved various manipulations of the oocyte. For example sperm have been injected directly into the egg with a high success rate [Fishel (1992) Lancet 339: 932-3]. However, since abnormal sperm may be injected by this method it is primarily limited by high genetic risks.

Only recently has attention turned to enhancement of sperm motility parameters. It has been shown that sperm motility parameters are important for both presenting the maximum number of male gametes to the egg as well as facilitating penetration through its zona pellucida. Sperm motility parameters have a high correlation with fertilization rates in vitro [Mahadevan and Trounson (1984) Fertil. Steril. 24: 131-4]. The only compound currently used to enhance sperm motility in vitro or in vivo is 3-7 dimethyl-1-5-oxohexylxanthine (pentoxifylline) whose efficacy remains questionable.

While reducing the present invention to practice the present inventor has uncovered that members of the ERK and p38 cascades are expressed in patches along the mid, principal and end pieces of a sperm cell tail, while absent from the sperm head, indicating a role for these proteins in sperm flagellar motility. Based on these findings the present inventor has found that specific inhibitors and/or activators of the p38 and ERK cascades can be used to modulate sperm motility and as such can be used as fertility and contraceptive medicaments.

Fertilization

According to one aspect of the present invention there is provided a method of enhancing fertility. The method is effected by providing to a subject a therapeutically effective amount of a p38 inhibitor and/or an ERK activator to thereby enhance fertility.

As is further described hereinbelow such a p38 inhibitor and/or an ERK activator can be applied topically (e.g., intra-vaginally) or administered systematically and as such can be provided to a female and/or a male individual.

Preferred male or female individual subjects according to this aspect of the present invention include mammals such as canines, felines, ovines, porcines, equines, bovines and humans.

As used herein a “p38 inhibitor” refers to a molecule and/or a condition which is capable of down-regulating or suppressing p38 activity or expression in sperm cells.

A p38 inhibitor according to this aspect of the present invention can be a p38 “direct inhibitor” which inhibits p38 intrinsic activity or expression (e.g., antisense, siRNA) or a p38 “indirect inhibitor” which inhibits the activity or expression of upstream components in the p38 signaling cascade (provided such upstream components are expressed in the sperm cell), thereby inhibiting p38 activity.

A p38 inhibitor molecule can be a natural negative regulator of p38 (e.g., Rap) or a p38 signaling molecule which has been modified to down-regulate or suppress p38 activity. Examples for such molecules include but are not limited to -dominant-negative p38 (e.g., p38βAGF), dominant-negative Rac [e.g., Rac(T17N)], dominant-negative cdc42 [e.g., cdc42(T17N)], dominant-negative MKK [e.g., MKK3(T193A), MKK4(S220A) and dominant negative MKK6), dominant-negative PAK1 (e.g., PAK1(K299R)], dominant-negative Ras [e.g., Ras(S17N) and Ras(S17W)]. For further details see Kuemmerle and Zhou, 2002 (J. Biol. Chem. 277: 20563-71), Verma et al., 2002 (J. Biol. Chem. 277: 44988-95), Hsu et al., 2002 (Blood Sep 5, electronic pub ahead of print), Zhang et al., 1995 (J. Biol. Chem. 270: 23934-6), Minden et al., 1994 (Science 266: 1719-1723), Yan et al., 1994 (Nature 372: 798-800), Raingeaud et al., 1996 (Mol. and Cell. Biol. 16: 1247-1255).

Alternatively, a p38 inhibitor molecule can be a chemical which is designed to specifically inhibit p38 activity. Non limiting examples of p38 inhibitors include 2-(4-Chlorophenyl)-4-(4-fluorophenyl)-5-pyridin-4-yl-1,2-dihydropyrazol-3-one, SC68376, SB203580(Iodo), SB202190, SB203580, SB203580(Sulfone), PD169316, SB220025, SKF-86002, SB239063, ML 3163 and thienyl urea analog (C₁₇H₂ON₂O₃S). Such inhibitors are available from a number of chemical companies including Calbiochem (San Diego, Calif., USA) and Sigma-Aldrich Corp. (St Louis, Mo., USA).

An inhibitor of p38 expression or of one of its upstream signaling component can be, for example, an antisense molecule designed and configured to inhibit p38 translation [Aoshiba et al. (1999) J. Immunol. 162(3): 1692-700]. Other inhibitor molecules can be neutralizing antibodies directed at each of the components in the p38 signaling pathway and the like.

As is mentioned hereinabove, the method according to this aspect of the present invention may employ an additional or alternative step of administering an ERK activator. As used herein an “ERK activator” refers to a molecule and/or a condition, which is capable of specifically up-regulating ERK activity or expression within a sperm cell. An ERK activator molecule can be, for example, any ERK cascade signaling molecule, which is located upstream of ERK in the signaling cascade (provided such upstream components are expressed in the sperm cell). These molecules may be wild type molecules or molecules which have been modified to be highly active (e.g., constitutively active). Examples of ERK activator molecules include but are not limited to: ERK1/2, Ras [e.g., Ras(G12V) and Ras(L61)], Raf-1 [e.g., Raf(BXB)], MEK1/2 [e.g., ΔN-EEMEK], PKC [e.g., 12-O-Tetradecanoylphorbol-13-acetate (TPA), diC8, OAG, arachidonic acid and the like]. For further details see Herman and Simonson, 1995 (J. Biol. Chem. 270: 11654-11661).

An ERK activating condition includes for example an ultra-violet (UV) radiation. Sperm cells are preferably exposed to UVB radiation at 280-320 nm. It will be appreciated though that due to the potential hazardous of UV radiation measures are taken not to over expose the sperm cells thereto.

Although each of the agents described hereinabove may enhance fertilization individually, it will be appreciated that for optimal activity, the method of the present invention preferably includes a combination of the above described fertility enhancing agents (i.e., p38 inhibitor and ERK activator). Other additives which may be used in conjunction with the agents of the present invention include for example, progesterone supplements, which are commonly used to enhance embryo implantation.

In cases wherein the p38 inhibitor and ERK activator of the present invention is a polypeptide, such as, for example, a signaling polypeptide, or a polynucleotide encoding same, preferably only the active portion of the signaling polypeptide (i.e., substrate binding, phosphorylation and signaling) is utilized as the p38 inhibitor and ERK activator to thereby avoid problems associated with synthesis of large proteins or polynucleotides.

Synthetic peptides can be prepared by classical methods known in the art, for example, by using standard solid phase techniques. The standard methods include exclusive solid phase synthesis, partial solid phase synthesis methods, fragment condensation, classical solution synthesis, and even by recombinant DNA technology. See, e.g., Merrifield, J. Am. Chem. Soc., 85: 2149 (1963), incorporated herein by reference. Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

Alternatively, the polypeptides of the present invention can be isolated from a biological source (e.g., a biological sample). Protein purification methods are well known in the art. Examples include but are not limited to fractionation of samples by ammonium sulfate precipitation and acid or chaotrope extraction. Exemplary purification steps may include hydroxyapatite, size exclusion, HPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Amersham-Pharmacia Biotech, UK), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulthydryl activation, hydrazide activation, and carboxyl and amino derivatives for carbodiimide coupling chemistries. These and other solid media are well known and widely used in the art, and are available from commercial suppliers. Selection of a particular method is preferably determined by the properties of the chosen support. See, for example, Affinity Chromatography: Principles & Methods, Pharmacia LKB Biotechnology, Uppsala, Sweden, 1988.

The polypeptides of the present invention can be isolated by exploitation of their biochemical, structural, and biological properties. For example, active ERK may be isolated using an anti-phosphorylated ERK antibody. Other methods of purification include purification of glycosylated proteins by lectin affinity chromatography and ion exchange chromatography [Methods in Enzymol., Vol. 182, “Guide to Protein Purification”, M. Deutscher, (ed.), Acad. Press, San Diego, 1990, pp.529-39].

To generate large amounts of the polypeptides of the present invention, recombinant techniques are preferably used.

Polynucleotides encoding the polypeptides of the present invention are first cloned into an appropriate expression vector (i.e., construct) examples of which are provided hereinunder.

Depending on the host/vector system utilized, any of a number of suitable transcription and translation elements including constitutive and inducible promoters, transcription enhancer elements, transcription terminators, and the like can be used in the expression vector [see, e.g., Bitter et al., (1987) Methods in Enzymol. 153:516-544].

Constructs encoding the polypeptides of the present invention are transformed into an appropriate host cell. Transformed cells are cultured under conditions, which allow for the expression of high amounts of recombinant polypeptide. Such conditions include, but are not limited to, media, bioreactor, temperature, pH and oxygen conditions that permit protein production. Media refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are well known to one of ordinary skill in the art.

Recovery of the recombinant polypeptide is effected following an appropriate time in culture. The phrase “recovering the recombinant polypeptide” refers to collecting the whole fermentation medium containing the polypeptide and need not imply additional steps of separation or purification. Not withstanding from the above, polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Although mature spermatozoa are fully differentiated cells which do not have active transcriptional machinery [Yanagimachi, R. (1994). Marnmalian fertilization. In: The Physiology of Reproduction, 2^{nd} edition, E. Knobil- and J. Neill, eds, pp. 189-317, Raven Press, New York], the proteins of the present invention can be expressed in premature spermatozoa from a nucleic acid construct administered to the subject employing any suitable mode of administration, described hereinbelow. Thus, although direct administration of a (recombinant) peptide inhibitor/activator is preferred, expression of such a peptide within a cell such as premature spermatozoa for the purpose of increasing motility of the mature spermatozoa is also envisaged by the present invention.

To enable cellular expression of the proteins of the present invention, the nucleic acid construct of the present invention further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any available promoter can be used by the present methodology. In a preferred embodiment of the present invention, the promoter utilized by the nucleic acid construct of the present invention is active in the specific cell population transformed. Examples of cell type-specific and/or tissue-specific promoters include promoters such as the TCP10 promoter [Rhee and Wolgemuth (2002). Mol. Cells 13: 85-90] or the human phosphoglycerate kinase 2 (PGK2) promoter [Tascou et al. (2001). Mol. Hum. Reprod. 7: 1123-1131].

The nucleic acid construct of the present invention can further include an enhancer, which can be adjacent or distant to the promoter sequence and can function in up regulating the transcription therefrom.

The constructs of the present methodology preferably further include an appropriate selectable marker and/or an origin of replication. Preferably, the construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome

Examples of suitable constructs include, but are not limited to pcDNA3, pcDNA3.1 (±), pGL3, PzeoSV2 (±), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pbabe, where the transgene will be transcribed from the 5′LTR promoter.

The fertility enhancing agent (active ingredient i.e., ERK activator, p38 inhibitor) of the present invention can be provided to an individual per se, or as part of a pharmaceutical composition where it is mixed with a pharmaceutically acceptable carrier.

Herein the term “active ingredient” refers to the preparation accountable for the biological effect or alternatively to a nucleic acid construct encoding same (in the case of a peptide inhibitor/activator).

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

As mentioned hereinabove, the pharmaceutical composition is formulated according to the individual treated and the site of application administration. For example, if the treated individual is a male, the pharmaceutical composition is preferably formulated as a topical cream.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections. Preferably, systemic administration, according to this aspect of the present invention is effected when male subject are treated.

Alternatively, one may administer a preparation in a local rather than systemic manner, for example, via administration of the preparation directly into a specific region of a patient's body such as to the subject genitalia. Thus, for example, the agents of the present invention can be administered intravaginally (i.e., into the female reproductive tract). This may be effected by application of the agents to the female genitalia prior to and/or concomitant with sexual contact, such as prior to sexual intercourse.

Formulations suitable for genital application include cream, ointment, lotion, jelly, solution, emulsion, spray or foam formulation containing a contraceptive effective amount of the agent.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

Formulations suitable for vaginal administration may be presented as pessaries, tampons, creams, gels, pastes, jelly, foams or sprays or aqueous or oily suspensions, solutions or emulsions (i.e., liquid formulations), or films containing in addition to the agents of the present invention, such carriers as are known in the art to be appropriate (described in details in U.S. Pat. No. 5,756,681).

The pH value of a transvaginal formulation for use in the present invention should preferably have a pH value close to that of the vagina, i.e., 3 to 7, preferably 4 to 6. The pH may be adjusted by an acid or base which is non-toxic and non-irritating to humans, for example, an organic acid such as acetic acid, or citric acid, or a weak base, such as sodium hydrogen carbonate or sodium acetate.

Compositions suitable for application to the vagina are disclosed in U.S. Pat. Nos.: 2,149,240, 2,330,846, 2,436,184, 2,467,884, 2,541,103, 2,623,839, 2,623,841, 3,062,715, 3,067,743, 3,108,043, 3,174,900, 3,244,589, 4,093,730, 4,187,286, 4,283,325, 4,321,277, 4,368,186, 4,371,518, 4,389,330, 4,415,585, 4,551,148, 4,999,342, 5,013,544, 5,227,160, 5,229,423, 5,314,917, 5,380,523, and 5,387,611.

For transurethral administration the composition contains one or more selected carriers excipients, such as water, silicone, waxes, petroleum jelly, polyethylene glycol (PEG), propylene glycol (PG), liposomes, sugars such as mannitol and lactose, and/or a variety of other materials, with polyethylene glycol and derivatives thereof. It is preferred that the pharmaceutical compositions contain one or more transurethral permeation enhancers, i.e., compounds which act to increase the rate at which the selected drug permeates through the urethral membrane. Examples of suitable permeation enhancers include dimethylsulfoxide (DMSO), dimethyl formamide (DMF), N,N-dimethylacetamide (DMA), decylmethylsulfoxide, polyethylene glycol monolaurate (PEGML), glycerol monolaurate, lecithin, the 1-substituted azacycloheptan-2-ones, particularly 1-n-dodecylcyclaza-cycloheptan-2-one (available under the trademark Azone®™ from Nelson Research & Development Co., Irvine, Calif.), SEPA®™ (available from Macrochem Co., Lexington, Mass.), alcohols (e.g., ethanol), surfactants including, for example, Tergitol®™, Nonoxynol-9®™ and TWEEN-80®™, and lower alkanols such as ethanol. As disclosed in WO91/16021, transurethral administration of an agent can be carried out in a number of different ways. For example, the agent can be introduced into the urethra from a flexible tube, squeeze bottle, pump or aerosol spray. The agent may also be contained in coatings, pellets or suppositories, which are absorbed, melted or bioeroded in the urethra. In certain embodiments, the agent is included in a coating on the exterior surface of a penile insert.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in a powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans. A preferred dose according to this aspect of the present invention is 10 mg/kg body weight.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fing], et al., 1975, in “The Pharmacological Basis of Therapeutics”, Ch. 1 p.1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Aside from the ability of the agents of the present invention to enhance fertilization in vivo, these agents can also be used to enhance sperm motility in vitro. The increased use of in vitro fertilization techniques by couples unable to bear children and artificial insemination programs for livestock benefit largely from agents, which enhance sperm motility parameters and, thus, lead to greater oocyte fertilization.

Thus, according to another aspect of the present invention there is provided a method of enhancing sperm motility. The method according to this aspect of the present invention is effected as follows.

First, a sperm sample (i.e., semen) is obtained. Semen can be collected by any method which is generally used for that species. For example, bovine and rabbit semen is typically collected by use of an artificial vagina. Human semen is typically collected by manual ejaculation. Sperm is preferably washed via centrifugation and may be frozen prior to use, provided the freezing techniques do not destroy viability. The sperm sample may be diluted as appropriate.

Thereafter, the sperm sample is contacted with an ERK activator and/or a p38 inhibitor (described hereinabove) to thereby enhance the motility of the sperm cells in the sperm sample. Preferably, incubation is effected at 37° C. under a CO₂ enriched atmosphere (e.g., 95% air and 5% CO₂). Incubation time may vary, preferably ranging from 20 to 60 minutes, such that maximal sperms within the semen sample will exhibit enhanced motility.

The concentration of the ERK activator and/or p38 inhibitor can also vary. Effective concentrations can be determined by those of skill in the art. Typically, the concentration is within a range of about (i.e., ±10%) 0.1 to 1.5 μg per μl semen suspension.

Following incubation, sperm is preferably washed with medium supplemented with 1% bovine serum albumin (BSA) although other additives may also be used.

Preferably, sperm cells which exhibit enhanced motility are isolated for future incubation with oocytes. Numerous methods for separating higher motile spermatozoa from lower motile spermatozoa are known in the art. Examples include but are not limited to the swim up, swim down and Percoll density gradients to obtain highly motile sperm populations. Motility may be defined by percentage of motile sperm versus immotile sperm and percentage of motile sperm with forward progression versus immotile sperm. Alternatively, the sperm motility index, which is a measurement of disturbances in optical density of the semen [Bartoov, et al. (1991) Fertil. Steril. 56:108-112] may be used

Motile sperm population can be incubated with oocytes either in vitro by artificial insemination or in vivo. Thus, eggs can be isolated from females and fertilized with the treated sperm prior to reimplantation. Alternatively, motile sperm can be incubated with eggs in vivo. Techniques for application in both instances are well known in the art.

As is further illustrated in Example 3 of the Examples section which follows, MAPK proteins may enhance the acrosome reaction. The acrosome reaction begins with the merging of the egg and sperm surfaces in which the content of the acrosome, including hydrolytic enzymes, are released. Thereafter, the plasma membrane of the acrosome disappears and the released hydrolytic enzymes facilitate the penetration of the acrosome through the egg membrane.

It will be appreciated that although MAPK proteins are absent from the sperm head their involvement in the acrosome reaction is likely to be mediated by other protein(s), possibly by ERK and p38 substrate(s), which upon phosphorylation in the sperm tail or neck migrate to the sperm head and participate in the acrosome reaction.

Therefore, it is highly conceivable that MAPK proteins may enhance fertilization by facilitating the acrosome reaction

For example, MAPK proteins or MAPK activators may be added to capacitated sperms during in vitro fertilization and thereby enhance the acrosome reaction and facilitate fertilization.

Contraception

Adequate sperm motility is known to be an essential prerequisite for successful fertilization. Sperm cells must possess vigorous motility to penetrate cervical mucus, migrate through the female reproductive tract to the site of fertilization and penetrate the oocyte vestments [Yanagimachi, R., (1994), Mammalian Fertilization, IN: Knobil, E. and Neill, J., (Eds.), Physiology of Reproduction, Raven Press, pp. 189]. Compounds that act on spermatozoa to interrupt one or more of these requisite events in the fertilization process provide unique means for contraceptive intervention.

As described hereinabove the present inventor has uncovered agents which affect sperm-cell motility and as such can serve as potent non-spermicidal contraceptives.

Thus, according to yet another aspect of the present invention there is provided a method of contraception. The method according to this aspect of the present invention is effected by providing to a subject an amount of a p38 activator and/or an ERK inhibitor which is capable of substantially reducing sperm motility.

As used herein the term “contraception” refers to the prevention of pregnancy.

As used herein the phrase “ERK inhibitor” refers to a molecule and/or a condition which is capable of down-regulating or suppressing ERK activity or expression in sperm cells.

An ERK inhibitor according to this aspect of the present invention can be an ERK “direct inhibitor” which inhibits ERK intrinsic activity or expression (e.g., antisense and siRNA) or an ERK “indirect inhibitor” which inhibits the activity or expression of upstream components in the ERK signaling cascade (provided such upstream components are expressed in the sperm cell), thereby inhibiting ERK activity.

An ERK inhibitor molecule can be a natural negative regulator of ERK or an ERK signaling molecule which has been modified to down-regulate or suppress ERK activity. Examples for such molecules include but are not limited to dominant-negative ERK [e.g., ERK1(K71R) and ERK2(K52R)], dominant-negative Ras [e.g., Ras(S17N) and Ras(S17W)] and dominant-negative Raf-1 [e.g., Raf-1(K375W), Raf(C4B), Raf 301 and Raf(S621A)] and dominant-negative MEK [e.g., MEK1(K97A), MEK1(K97M), MEK2(K101A) and MEK1/2(KAMEK)], as is further detailed elsewhere [Cheng et al., 1999, JBC 274: 6553-8; Chen et al., 2000, Mol. Pharmacol. 57:-153-61; Salomoni et al;, 1998, J. Exp. Med. 187: 1995-2007; Sun et al., 2002, J. Biol. Chem. 277: 1544-1552; Minden et al., 1994, Science 266: 1719-1723; Coso et al., 1995, Cell 81: 1137-1146; Bruder et al., 1992, Genes and Develop. 6: 545-556; Qureshi et al., 1991, J. Biol. Chem. 266: 20594-20597; Abbott and Holt, 1999, J. Biol. Chem. 274: 2732-2742; Gardner et al., 1994, Mol. Biol. Cell 5:193-201].

Alternatively, an ERK inhibitor molecule can be a chemical or a peptide which is designed to specifically inhibit ERK activity. Non limiting examples of ERK specific inhibitors include PD98059, PD184352, U0126, ITU, Ste-MEK1₁₃, MTP_{TAT}-G-MEK1₁₃. Examples of MEK inhibitors include but are not limited to PD98059, U0126 and U0125. Examples of Raf-1 inhibitors include but are not limited to Raf-1 kinase inhibitor I (5-Iodo-3[(3,5-dibromo4 hydroxyphenyl) methylene]-2 indolinone) and ZM 336372. An example of Ras inhibitor is Ras inhibitory peptide such as set forth in VPPPVPPRRR (Calbiochem, San Diego, Calif., USA). Examples of PKC inhibitors include but are not limited to GF109203X, PKC 19-31, PKC 19-36, staurosporine and G06976. Such inhibitors are available from a number of chemical companies including Calbiochem (San Diego, Calif., USA) and Sigma-Aldrich Corp. (St Louis, Mo., USA).

As is mentioned hereinabove, the method according to this aspect of the present invention may employ an alternative or additional step of administering a p38 activator. As used herein a “p38 activator” refers to a molecule and/or a condition which is capable of specifically up-regulating p38 activity or expression within a sperm cell. A p38 activator molecule can be any p38 cascade signaling molecule which is located upstream of p38 in the signaling cascade (provided such upstream components are expressed in the sperm cell). These molecules may be wild type molecules or molecules which have been modified to be highly active to thereby generate strong signals (e~g., constitutive activity). Examples of p38 activator molecules include but are not limited to: a p38 activating growth factor (e.g., IL-1, constitutively active IL-1 receptor, TNF, LPS, TRAF6 and TAB1/2), MKK (e.g., constitutively active MKK3, constitutively active MKK4 and constitutively active MKK6), Rac [e.g., Rac(V12) and Rac(L61)], Cdc42 [e.g., Cdc42(Q61L) and Cdc42(V12)] and PAK1.

A p38 activating condition includes for example application of a physical force (e.g., tensile forces through collagen-coated magnetite beads), application of a chemical stress (e.g., sodium arsenite) and an osmotic shock (e.g., exposure to hyperosmotic media, sorbitol or NaCl) as is further described elsewhere [D'Addario et al., 2002, J. Biol. Chem. 277: 47541-50; Werz et al., 2001, J. Leukoc. Biol. 70: 830-8; Kishi et al., 2001, J Biol Chem 276: 39115-22)].

The p38 activator/ERK inhibitor of this aspect of the present invention can be administered per se, or as a part of a pharmaceutical composition. Formulations of pharmaceutical composition suitable for contraceptive use are described hereinabove.

The contraceptive agents of the present invention can be utilized in conjunction with a contraceptive device by applying the agents on a contraceptive device (e.g., a condom, a contraceptive diaphragm or a contraceptive sponge, such as, a collagen sponge or a polyurethane foam sponge), prior to sexual intercourse. Alternatively, agents of the present invention can be applied on a pessary or tampon for vaginal administration.

The amount of the contraceptive agents of this aspect of the present invention can vary depending also on the route of administration and the age and condition of the subject. For example, the preferred dosage of the contraceptive agent formulated in a pharmaceutical composition is 10 mg/kg body weight.

According to another preferred embodiment of the present invention the contraceptive agent is included in a topical preparation. A preferred concentration of the agent in a topical dosage varies from 100 to 500 μg/ml such that effective immobilization of the sperm present in the vagina is achieved and its penetration in the cervical mucus is inhibited.

The contraceptive agents of the present invention are preferably co-administered with additional contraceptives, which may either act in a spermicidal or non-spermicidal way. Examples of such additional contraceptives include nonylphenoxypolyoxyethylene glycol (monoxynol-9), benzethonium chloride and chlorindanol.

As is further illustrated in Example 3 of the Examples section which follows the present inventor has uncovered that MAPK inhibitors may also inhibit the acrosome reaction.

Thus, according to this aspect of the present invention, inhibitors of MAK proteins may be used as potent contraceptive by inhibiting both motility and capacitation of sperm cells.

The strong correlation between sperm motility and sperm quality motivated the development of numerous assays for evaluating semen quality. In one such assay, sperm cells from a test specimen are allowed to “swim-up” into a clear medium from a concentrated sperm suspension at the bottom of an optical cuvette. Highly motile sperm cause a time-dependent increase in turbidity of the medium, which can be used to determine a fraction of rapidly moving sperm and an average velocity of the sperm. The changing turbidity of the medium is recorded by a spectrophotometer as an increase in absorbance. However, despite wide use thereof, these methods are laborious, time consuming and require high professionalism from the lab technicians.

As described hereinabove the present inventor established a strong correlation between p38 activity and sperm quality. Thus, according to another aspect of the present invention there is provided a method of determining quality of a semen sample.

The method according to this aspect of the present invention includes determining the activity levels of p38 in the sperm cells of the semen sample, which activity is inversely correlated with sperm cell motility, thereby determining the quality of the semen sample.

There is a wide range of assays which can be used to measure p38 kinase activity. The phosphorylation state of the p38 kinase is correlated with its activation.

Thus, activation of p38 kinase can be measured by determining the level of phosphorylation thereof. This can be done by allowing phosphorylation to occur in the presence ³²P-ATP or some other detectable label. Activity of the p38 kinase can also be determined by measuring its ability to phosphorylate a substrate such as MEF2C and myelin-basic-protein (MBP) in an SDS-gel [Wang et al., (1992). Mol. Biol. Cell 3: 1329].

Alternatively, an antibody specific for the phosphorylated form of p38 kinase (e.g., M8177 Sigma-Aldrich Co.) can be used to measure p38 kinase activity by measuring the level of phosphoiylated p38 kinase using Western blotting or an ELISA assay or other antibody based protein detection and quantification assays.

Alternatively, a reporter assay may be used to determine p38 activity. Briefly, a reporter gene encoding any detectable gene product whose expression is related to the ability of p38 to effect expression of the gene construct is selected. The transcriptional control elements are p38-specific, and therefore, any effect measured by expression of the reporter gene indicates an effect on the p38 pathway. Reporter assays and other methods of detecting p38 kinase activity are disclosed in U.S. Pat. No. 6,010,856.

It will be appreciated that the active agents (i.e., contraceptive agents, fertility enhancing agents and diagnostic agents) described herein can also be included in kits. For example a diagnostic kit for assessing quality of a sperm sample can include an antibody directed at the phosphorylated form of p38 (such as described above) in a one container and a solid phase for attaching multiple sperm samples packaged in a second container with appropriate buffers and preservatives and used for diagnosis.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D.; and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

The Mitogen Activated Protein Kinase (MAPK) Cascade

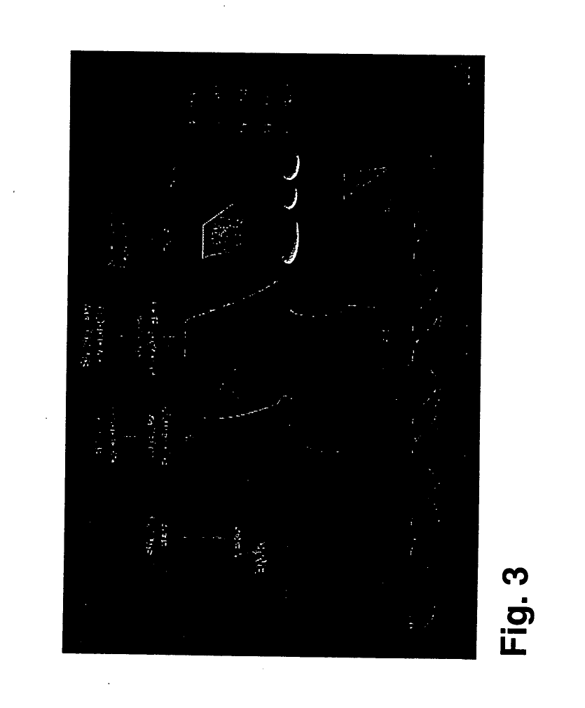

Mitogen-activated protein kinases (MAPK) are a family of proline-directed serine/threonine kinases that activate their substrates by phosphorylation. The MAPK family consists of key regulatory proteins that are known to regulate cellular responses to both proliferative and stress signals. There are three distinct groups of MAPKs in mammalian cells: a) extracellular signal-regulated kinases (ERKs), b) c-Jun N-terminal kinases (JNKs) and c) stress activated protein kinases (SAPKs) (for further details see FIG. 3 and Seger and Krebs, 1995).

The MAP kinases are activated by a variety of signals including nutritional and osmotic stress, UV light, growth factors, endotoxin, inflammatory cytokines and hormones. For example, PKC activation, leads to Raf-1 activation, which further activates the MAPK/ERK kinases (MEKs). MEKs activate ERKs which translocate to the cell nucleus where they activate transcription factors and thereby regulate cell proliferation.

Recent reports have suggested the involvement of MAPK cascade proteins in spermatogenesis and spermatozoa maturation (Lu et al., 1999; Luconi et al., 1998). To substantiate a role for MAPK proteins in sperm function the present inventor has conducted a comprehensive set of experiments to refine the intra-cellular localization of the MAPK cascade proteins in human spermatozoa and elucidate their involvement in sperm function.

### Example 1

### The MAPK Cascade Proteins are Localized in the Sperm Neck and Tail but not the Sperm Head

To localize the MAPK proteins in human spermatozoa antibodies directed at the MAPK cascade proteins were employed on human spermatozoa.

Materials and Experimental Methods

Isolation of human sperm samples—Human semen were obtained upon informed consent from healthy donors having normal sperm density, motility and morphology (according to the World Health Organization (WHO) guidelines of 1992) following 3 days of sexual abstinence. Sperm samples were collected into sterile 50 ml tubes and semen was liquefied for 1 hour at 36° C., washed twice with HAM's F-10 medium (Biological Industries, Beth Ha'Emek, Israel) supplemented with 0.3% bovine serum albumin (BSA) and separated from non-sperm cells by a Percoll gradient centrifugation (Sigma-Aldrich Corp., St Louis, Mo., USA) essentially as described elsewhere (Rotem et al., 1990a, b). The resultant sperm sample contained approx. 50×10⁶ sperm/ml.

Sperm capacitation—Isolated sperm samples were incubated for 3 hours at 36° C. in HAM's F-10 medium supplemented with 0.3% BSA as in Rotem et al., 1990a, b

Western blot analysis—Western blot analysis was effected with antibodies directed at ERK, JNK and p38 proteins as well as with antibodies directed at the phosphorylated forms of these MAPK proteins (Sigma Rehovot, Israel). The assay was performed as previously described (Yung et al., 1997).

Immunohistochemistry (IHC)—Sperm samples were cyto-spun for 10 min at 600 RPM. Cyto-spin slides were fixed in cold methanol (100%) followed by incubation with cold acetone (100%) for 10 min each. Fixed samples were washed three times in phosphate buffered saline (PBS), 5 min each, incubated in 0.3% H₂O₂ in PBS for 20 min and washed three times in PBS, 5 min each. Slides were then blocked for 30 min in 3% fetal calf serum in PBS and incubated for overnight at 4° C. with 1:25 dilutions of anti ERK1/2 (Sigma, Rehovot, Israel), anti p38 (α1/2, Sigma, Rehovot, Israel), anti MEK1 (Santa Cruz, Calif., USA), anti m-SOS (Santa Cruz, Calif., USA), anti Raf-1 (Santa Cruz, Calif., USA) and anti tubulin (prepared by Dr. I. Gozes, Tel Aviv University, Israel). Slides were then washed three times in PBS, 20 min each, and further incubated for 45 min with 1:100 dilutions of biotin conjugated anti rabbit IgG antibodies. Samples were further washed three times in PBS, 10 min each, and incubated for 30 min with avidin peroxidase complex as described by Kalina et al. (1995). Bound antibodies were visualized using diaminobenzidine (0.25 mg/ml) and 0.01% H₂O₂ in PBS as described by Kalina et al. (1995).

Immunoelectron microscopy—Cells were fixed with 2% glutaraldehyde in 0.1 M cacodylate buffer for 60 min at room temperature. Cells were then washed in PBS, dehydrated in acetone and embedded in araldite as described by Kalina et al. (1995). Sections were placed on silver grids, permeabilized with 0.1% Triton X-100 in PBS for 20 min, washed in PBS and blocked for 1 hour in 1% BSA in PBS. Sections were incubated for overnight at 4° C. with 1:50 dilutions of anti ERK antibody. Sections were then washed in 0.05 M Tris-buffered saline (TBS, pH 7.3) and incubated for 1 hour with a 1:10 dilution of a secondary 8-nm gold-conjugated goat anti rabbit IgG (Biocell, Cardiff, UK) in TBS (pH 8.4) supplemented with 1% egg albumin. Sections were further rinsed three times in TBS, 5 min each, and subjected to contrast enhancement using uranyl acetate and lead citrate as described by Kalina et al. (1995). Sections were examined and photographed using the JEOL 100B (JEOL Inc. Peabody, Mass., USA) electron microscope apparatus.

Experimental Results

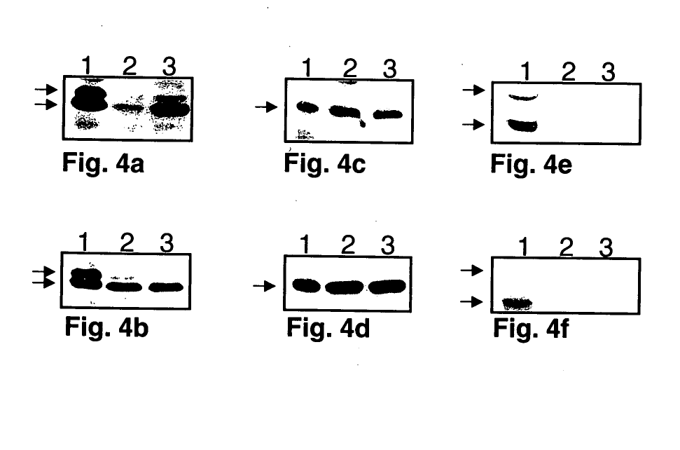

Molecular identification of MAPK cascade proteins in human spermatozoa—Capacitated human spermatozoa cell lysate were generated as described and subjected to Western blot analysis using MAPK antibodies specific to either the phosphorylated or the general forms of ERK, p38 and INK proteins. Efficacy of antibodies was first tested on a control cell lysate prepared from the LβT2 pituitary cell line following activation with TPA (50 ng/ml) for 60 min. Positive binding signals confirmed the specificity of the phosphorylated, active—and the unphosphorylated, general forms of ERK, p38 and JNK (FIGS. 4a-f, lane 1). Similar binding signals were observed in human spermatozoa using the antibodies against the ERK (FIGS. 4a, b) and p38 (FIGS. 4c, d) but not the antibodies against JNK (FIGS. 4e, f). These results suggest that ERK and p38 but not JNK are present in capacitated human spermatozoa. In addition, while the phosphorylated form of p38 was detected in capacitated human spermatozoa regardless of vanadate treatment (FIG. 4c, lanes 2 and 3) the phosphorylated ERK could be detected only following treatment with the tyrosine phosphatase inhibitor, vanadate (FIG. 4a, lane 2 and 3).

Determination of phosphorylated ERK by in-gel kinase assay—To further substantiate its role in spermatozoa maturation the phosphorylation state of ERK was determined by in-gel kinase assay as described elsewhere (Naor et al., 1999). Human spermatozoa, stimulated by progesterone (5 μg/ml) exhibited two phosphorylated bands of 44 and 42 kDa corresponding to the ERK 1 and ERK 2 proteins, respectively (not shown). These results suggest that both ERK proteins [Pearson G, Robinson F, Beers-Gibson T, Xu B-E, Karandikar M, Berman K, Cobb MH (2001). Mitogen-activated protein (MAP) kinase pathways: regulation and physiological functions. Endocr. Rev. 22: 153-183] are active in human spermatozoa.

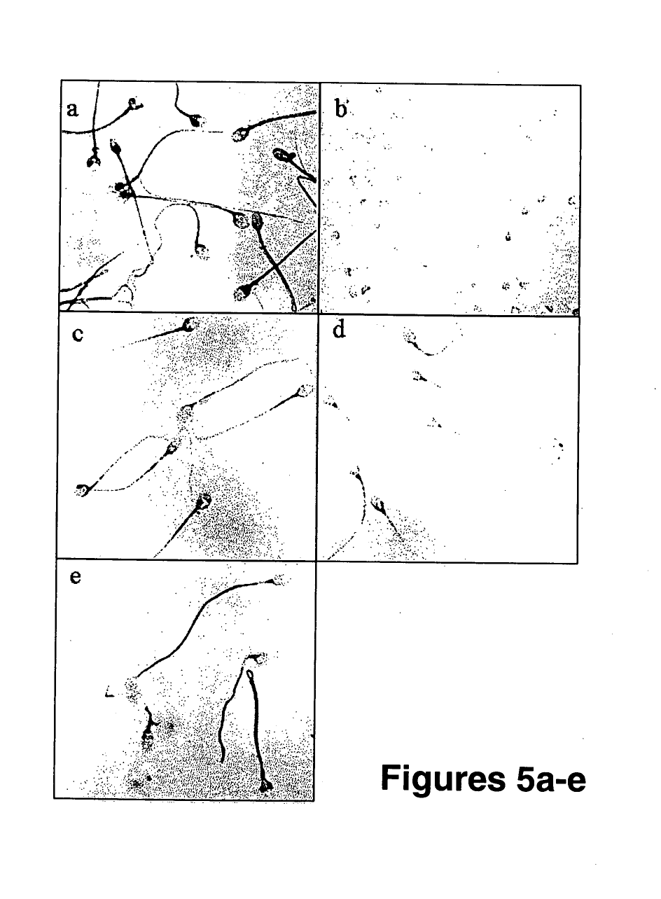

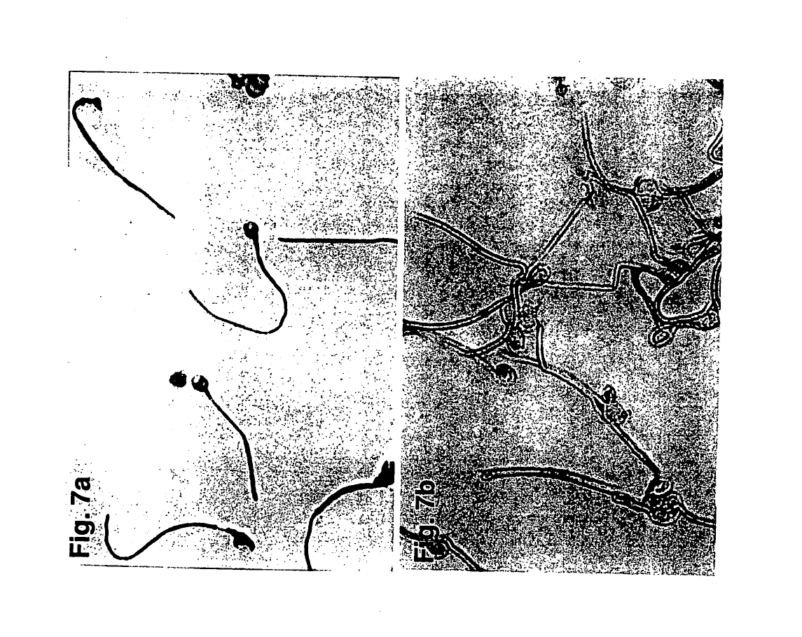

Intra-sperm localization of MAPK proteins—THC analyses localized the ERK, Raf-1, MEK1, p38 and mSOS proteins in the sperm neck and in patches along the mid, principle and end pieces of the sperm (FIGS. 5, 7 and data not shown). On the other hand, IHC using an anti tubulin antibody resulted in a continuous staining along the entire length of the tail (FIG. 5e). In contrast to previous publications by Luconi et al. [Luconi M. Krausz C, Bami T, Vannelli G B, Forti G, Baldi E (1998). Progesterone stimulates p42 extracellular signal regulated kinase (p42 ERK) in human spermatozoa. Mol. Hum. Reprod. 4: 251-258], and Baldi et al. [Baldi E, Luconi M, Bonaccorsi L, Forti G (1998). Nongenomic effects of progesterone on spermatozoa: mechanisms of signal transduction and clinical implications. Front Biosci. 3: D1051-D1059], the ERK and p38 were not expressed in the sperm head and the observed staining in the sperm head was not-specific as could be seen in samples treated with pre-absorbed antibodies (FIG. 5b and FIG. 7b, respectively).

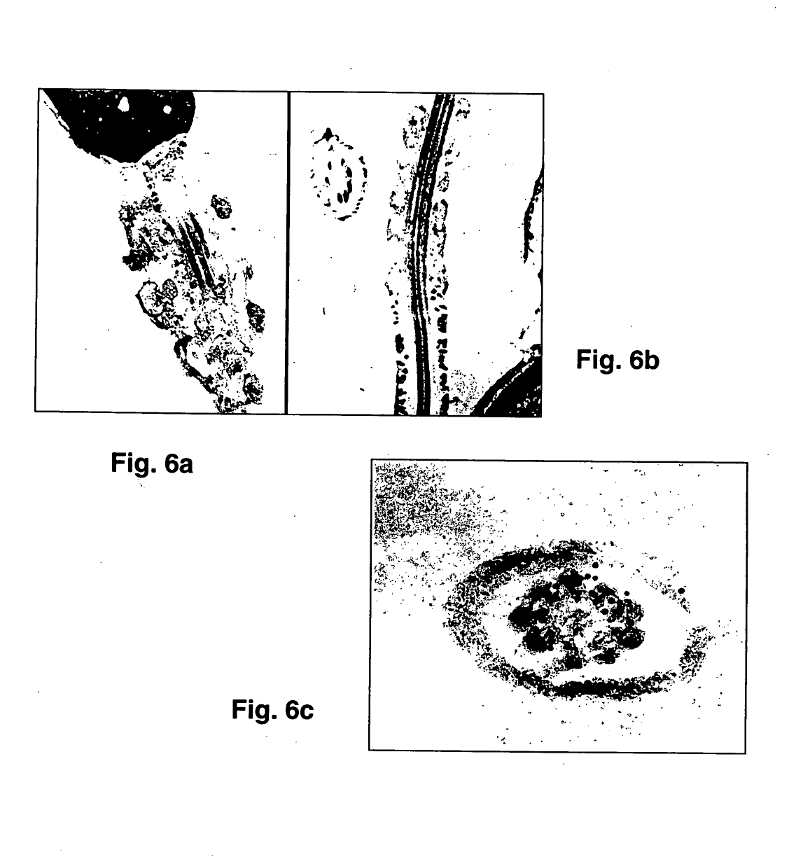

High power localization of ERK in human sperm—To further substantiate the involvement of ERK in spermatozoa functions, immuno-electron microscopy was employed. Gold-labeled antibodies revealed ERK labeling in a segmented column of the sperm neck and the outer dense fibers (FIG. 6a) and in the outer dense fibers and the axoneme in the mid and principle pieces (FIGS. 6b-c). No ERK labeling was observed in the mitochondria of both the sperm neck and the mid piece (FIGS. 6a and 6b, respectively) and in the fibrous sheath of the principal piece (FIG. 6b).

Altogether, these results demonstrate that ERK and p38, but not JNK, are present as either phosphorylated or unphosphorylated kinases in human spermatozoa and are localized in the sperm neck and tail but not the sperm head. Therefore, these results suggest that these MAPK proteins can participate in processes involved in sperm motility, such as flagellar function.

### Example 2

### The Effects of MAPK Inhibitors on Sperm Motility

To further elucidate the possible role of the MAPK cascade proteins in sperm motility specific MAPK inhibitors were added to capacitated human spermatozoa and sperm motility was determined.

Materials and Experimental Methods

Drug effect on sperm motility—To test the effect of MAPK inhibitors and progesterone on sperm motility the following drugs were added to the incubation medium: PD98059 (ERK inhibitor, Biomol, Plymouth Meeting, Pa., USA), SB203580 (p38 inhibitor, Biomol, Plymouth Meeting, Pa., USA) and progesterone (Sigma-Aldrich Corp., St Louis, Mo., USA). Progressive flagellar motility was determined as described elsewhere (Rotem et al., 1990a, b).

Experimental Results

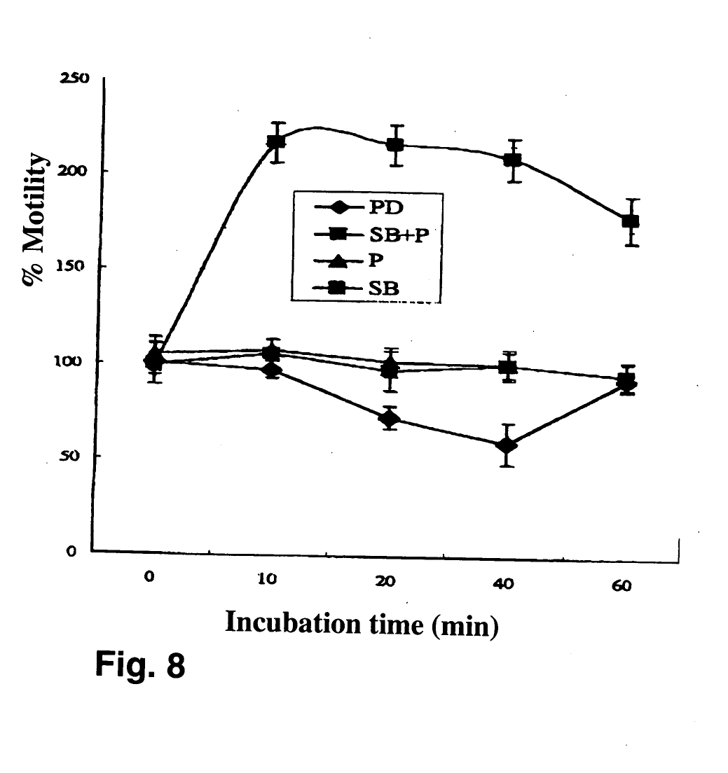

MAPK inhibitors affect sperm motility—Sperm motility was measured following capacitation in the presence of various drugs and inhibitors. Incubation of human spermatozoa in the presence of 5 μg/ml progesterone had no effect on the progressive flagellar motility (FIG. 8, marked with “P”). However, when human spermatozoa were incubated in the presence of 50 μM of the selective p38 inhibitor, SB203580, a significant increase of over 120% in the progressive flagellar motility was observed (FIG. 8, marked with “SB”). This increase was completely abolished when SB203580 was co-introduced to human spermatozoa with 5 μg/ml progesterone (FIG. 8, marked with “SB+P”). On the other hand, when incubation of human spermatozoa was in the presence of 50 μM of the MEK/ERK inhibitor, PD98059, the progressive flagellar motility was transiently decreased to 50% (FIG. 8, marked with “PD”).

Altogether these results demonstrate the involvement of p38 and ERK/MEK in sperm flagellar motility and suggest the use of p38 inhibitors and ERK activators as sperm motility enhancing drugs and the use of ERK inhibitors and p38 activators as sperm motility inhibiting drugs.

### Example 3

### The Effects of MAPK Inhibitors on Acrosome Reaction

To test the possible involvement of MAPK cascade proteins in the acrosome reaction various MAPK inhibitors were added to capacitated human spermatozoa and the acrosome reaction was determined.

Materials and Experimental Methods

Drug effect on acrosome reaction—To test the involvement of MAPK cascade proteins on acrosome reaction the following drugs were added to the incubation medium: Progesterone (Sigma-Aldrich Corp., St Louis, Mo., USA), hZP-3 (human zona pellucida protein 3, obtained from P. Salling, N.C., USA) diluted in MOCK (F-10 medium containing 0.1% DMSO, Biological Industries, Beth Ha'Emek, Israel), A23187 (calcium ionophore, Calbiochem, La Jolla, Calif., USA), PD98059 (Biomol, Plymouth Meeting, Pa., USA), SB203580 (Biomol, Plymouth Meeting, Pa., USA) and GF109203X (PKC inhibitor, Calbiochem, La Jolla, Calif., USA). Acrosome reaction was determined as described elsewhere (Rotem et al., 1992).

Experimental Results

MAPK inhibitors affect acrosome reaction—Acrosome reaction (AR) was stimulated by the following inducers: progesterone (50%), human zona pellucida protein 3 (120%) and A23187 (140%). A slight and non-specific increase in the acrosome reaction was also observed in the presence of MOCK, the ZP-3 diluent medium. The stimulated AR observed in the presence of progesterone and hZP-3 was markedly inhibited by GF109203X, SB203580 and PD098059 (Table 1, hereinbelow). However, the non-specific increase in AR stimulated by MOCK was inhibited only in the presence of SB203580. On the other hand, the AR stimulated by A23187 persisted in the presence of GF109203X, SB203580 and PD98059 (Table 1, hereinbelow).

TABLE 1
Role of MAPK in acrosome reaction

| | | GF109203X | SB203580 | PD98059 |
|---|---|---|---|---|
| | Control | (100 nM) | (20 μM) | (5 μM) |
| Control | 21 | 19 | 17 | 17 |
| Progesterone | 31 | 15 | 11 | 18 |
| 5 μg/ml | | | | |
| hZP3 | 47 | 18 | 28 | 29 |
| 50 ng/ml | | | | |
| MOCK | 30 | 28 | 15 | 25 |
| A23187 | 51 | 48 | 49 | 50 |
| 10 μM | | | | |

Table 1: Capacitated human spermatozoa were incubated with progesterone, human zona pellucida protein 3 (hZP3), MOCK or the calcium ionophore A23187, in the presence or absence of the PKC inhibitor (GF109203X), the p38 inhibitor (SB203580) or the MEK/ERK inhibitor (PD98059). Acrosome reaction was determined as previously described (Rotem et al., 1992).

These results demonstrate that ERK and p38 participate in the AR and that their inhibition by PD98059 and SB203580, respectively can inhibit the AR. However, since ERK and p38 are not present in the sperm head, their possible involvement in the acrosome reaction is likely to be mediated by other protein(s), possibly by ERK and p38 substrate(s), which upon phosphorylation in the sperm tail or neck migrate to the sperm head and participate in the acrosome reaction. Therefore, these results suggest the use of ERK and p38 inhibitors as AR inhibiting drugs, and the use of ERK and p38 activators as AR enhancing drugs.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED

### Additional References are Cited in the Text

1. Eisenbach, M., Tur-Kaspa, I. (1999). Do human eggs attract spermatozoa. Bioassays 21: 203-210.
2. Kalina, M., Socher, R., Rotem, R., Naor, Z. (1995). Ultrastractural localization of protein kinase C in human sperm. J. Histochem. Cytochem. 43: 439-445.
3. Lu, Q., Sun, Q. Y., Breitbart, H., Chen, D. Y. (1999). Expression and phosphorylation of mitogen activated protein kinases during spermatogenesis and epididymal sperm maturation in mice. Archives of Androl. 43: 55-66.
4. Luconi, M., Kraus, Z. C., Barni, T., Vannelli, G. B., Forti, G., Baldi, E. (1998). Progesterone stimulates p42 extracellular signal regulated kinase (p42 ERK) in human spermatozoa. Mol. Human Reprod. 4: 251-258.
5. Naor, Z., Benard, O., Seger, R. (1999). Activation of MAPK cascades by G-protein coupled receptors: the case of gonadotropin releasing hormone receptor. Trends Endocrinol. Metab. 11: 91-99.
6. Rotem, R., Paz, G. F., Homonnai, Z. T., Kalina, M., Naor, Z. (1990a). Protein kinase C is present in human sperm: possible role in flagellar motility. Proc. Nati. Acad. Sci. USA. 87: 7305-7308.
7. Rotem, R., Paz, G. F., Homonnai, Z. T., Kalina, M., Naor, Z. (1990b). Further studies on the involvement of protein kinase C in human sperm flagellar motility. Endocrinology 127: 2571-2577.
8. Rotem, R., Paz, G. F., Homonnai, Z. T., Kalina, M., Lax, J., Breitbart, H., Naor, Z. (1992). Ca²⁺ independent induction of acrosome reaction by protein kinase C in human sperm. Endocrinology 131: 2235-2243.
9. Seger, R., Krebs, E. G. (1995). The MAPK signaling cascade. FASEB. J. 9: 726-735.
10. Yanagimachi, R. (1994). Mammalian fertilization. In: The Physiology of Reproduction, 2^{nd} edition, E. Knobil and J. Neill, eds, pp. 189-317, Raven Press, New York.
11. Yung, Y., Dolginov, Y., Yao, Z., Rubinfeld, H., Michael, D., Hanoch, T., Roubini, E., Lando, Z., Zharhary, D., Seger, R. (1997). Detection of ERK activation by a novel monoclonal antibody. FEBS Lett. 408: 292-296.
12. Kamischke, A., Heuermann, T., Kruger, K., von Eckardstein, S., Schellschmidt, I., Rubig, A., Nieschlag, E. (2002). An effective hormonal male contraceptive using testosterone undecanoate with oral or injectable norethisterone preparations. J Clin. Endocrinol. Metab. 87: 530-9.

1. A method of contraception comprising providing to a subject an amount of a p38 activator and/or an ERK inhibitor capable of substantially reducing sperm motility.

2. The method of claim 1, wherein said subject is a female and said step of providing is effected via intravaginal administration of said p38 activator and/or ERK inhibitor.

3. The method of claim 1, wherein said subject is a male and said step of providing is effected via genital administration of said p38 activator and/or ERK inhibitor.

4. The method of claim 1, wherein said p38 activator is selected from the group consisting of p38 activating growth factor, MKK, Rac, Cdc42 and PAK1.

5. The method of claim 4, wherein said p38 activating growth factor is selected from the group consisting of IL-1, IL-1-receptor, TNF, LPS, TRAF6 and TAB1/2.

6. The method of claim 4, wherein said MKK is selected from the group consisting of MKK3, MKK4 and MKK6.

7. The method of claim 4, wherein said Rac is selected from the group consisting of Rac(V12) and Rac(L61).

8. The method of claim 4, wherein said Cdc42 is selected from the group consisting of Cdc42(Q61L) and Cdc42(V12).

9. The method of claim 1, wherein said p38 activator is a condition selected from the group consisting of physical stress, chemical stress and osmotic shock.

10. The method of claim 1, wherein said ERK inhibitor is selected from the group consisting of a PKC inhibitor, a Ras inhibitor, a dominant negative Ras, a Raf-1 inhibitor, a dominant negative Raf-1, a MEK inhibitor, a dominant negative MEK, a dominant negative ERK: and an ERK inhibitor.

11. The method of claim 10, wherein said PKC inhibitor is selected from the group consisting of GF109203X, PKC 19-31, PKC 19-36, staurosporine and GO 6976.

12. The method of claim 10, wherein said Ras inhibitor is a Ras inhibitory peptide.

13. The method of claim 10, wherein said dominant negative Ras is selected from the group consisting of Ras(S17N) and Ras(S17W).

14. The method of claim 10, wherein said Raf-1 inhibitor is selected from the group consisting of Raf-1 kinase inhibitor I (5-Iodo-3[(3,5-dibromo-4 hydroxyphenyl) methylene]-2 indolinone) and ZM 336372.

15. The method of claim 10, wherein said dominant negative Raf-1 is selected from the group consisting of Raf-1(K375W), Raf(C4B), Raf 301 and Raf(S621A).

16. The method of claim 10, wherein said MEK inhibitor is selected from the group consisting of PD98059, U0126 and U0125.

17. The method of claim 10, wherein said dominant negative MEK is selected from the group consisting of MEK1(K97A), MEK1(K97M), MEK2(K101A) and MEK1/2(KAMEK).

18. The method of claim 10, wherein said dominant negative ERK is selected from the group consisting of ERK1(K71R) and ERK2(K52R).

19. The method of claim 10, wherein said ERK inhibitor is selected from the group consisting of PD98059, PD184352, U0126, ITU, Ste-MEK1₁₃ and MTP_{TAT}-G-MEK1 ₁₃.

20. The method of claim 1, wherein said amount of p38 activator and/or ERK inhibitor does not exceed 10 mg/kg body weight.

21. A method of enhancing fertility comprising providing to a subject a therapeutically effective amount of a p38 inhibitor and/or an ERK activator, thereby enhancing fertility.

22. The method of claim 21, wherein said subject is a female and said step of providing is effected via intravaginal administration of said p38 inhibitor and/or ERK activator.

23. The method of claim 21, wherein said subject is a male and said step of providing is effected via genital administration of said p38 inhibitor and/or ERK activator.

24. The method of claim 21, further comprising providing to said subject a therapeutically effective amount of progesterone.

25. The method of claim 21, wherein said p38 inhibitor is selected from the group consisting of Rap, a dominant negative p38, a dominant negative Rac, a dominant negative Cdc42, a dominant negative MKK, a dominant negative Ras, a dominant negative PAK1 and a p38 inhibitor.

26. The method of claim 25, wherein said dominant negative p38 is p38betaAGF.

27. The method of claim 25, wherein said dominant negative Rac is Rac(T17N).

28. The method of claim 25, wherein said dominant negative Cdc42 is Cdc42(T17N).

29. The method of claim 25, wherein said dominant negative MKK is selected from the group consisting of MKK3(T193A), MKK4(S220A) and a dominant negative MKK6.

30. The method of claim 25, wherein said dominant negative Ras is selected from the group consisting of Ras(S17N) and Ras(S 17W).

31. The method of claim 25, wherein said dominant negative PAK1 is PAKI (K299R).

32. The method of claim 25, wherein said p38 inhibitor is selected from the group consisting of 2-(4-Chlorophenyl)-4-(4-fluorophenyl)-5-pyridin-4-yl- 1,2-dihydropyrazol-3-one, SC68376, SB203580(Iodo), SB202190, SB203580, SB203580(Sulfone), PD169316, SB220025, SKF-86002, SB239063, ML 3163 and a thienyl urea analog (C₁₇H₂ON₂O₃S).

33. The method of claim 21, wherein said ERK activator is selected from the group consisting of Ras, Raf-1, MEK1/2, a PKC activator and UV induction.

34. The method of claim 33, wherein said Ras is selected from the group consisting of Ras(G12V) and Ras(L61).

35. The method of claim 33, wherein said Raf-1 is Raf(BXB).

36. The method of claim 33, wherein said MEK1/2 is ΔN-EEMEK.

37. The method of claim 33, wherein said PKC activator is selected from the group consisting of TPA, diC8, OAG and arachidonic acid.

38. The method of claim 21, wherein said amount of p38 inhibitor and/or ERK activator does not exceed 10 mg/kg body weight.

39. Use of an ERK activator and/or a p38 inhibitor for the manufacture of a medicament for enhancing fertility.

40. The use of claim 39, wherein said ERK activator is selected from the group consisting of Ras, Raf-1, MEK1/2, a PKC activator and UV induction.

41. The use of claim 40, wherein said Ras is selected from the group consisting of Ras(G12V) and Ras(L61).

42. The use of claim 40, wherein said Raf-1 is Raf(BXB).

43. The use of claim 40, wherein said MEK1/2 is ΔN-EEMEK.

44. The use of claim 40, wherein said PKC activator is selected from the group consisting of TPA, diC8, OAG and arachidonic acid.

45. The use of claim 39, wherein said p38 inhibitor is selected from the group consisting of Rap, a dominant negative p38, a dominant negative Rac, a dominant negative Cdc42, a dominant negative MKK, a dominant negative Ras, a dominant negative PAK1 and a p38 inhibitor.

46. The use of claim 45, wherein said dominant negative p38 is p38betaAGF.

47. The use of claim 45, wherein said dominant negative Rac is Rac(T17N).

48. The use of claim 45, wherein said dominant negative Cdc42 is Cdc42(T17N).

49. The use of claim 45, wherein said dominant negative MKK is selected from the group consisting of MKK3(T193A), MKK4(S220A) and a dominant negative MKK6.

50. The use of claim 45, wherein said dominant negative Ras is selected from the group consisting of Ras(S17N) and Ras(S17W).

51. The use of claim 45, wherein said dominant negative PAK1 is PAK1(K299R).

52. The use of claim 45, wherein said p38 inhibitor is selected from the group consisting of 2-(4-Chlorophenyl)-4-(4-fluorophenyl)-5-pyridin-4-yl- 1,2-dihydropyrazol-3-one, SC68376, SB203580(Iodo), SB202190, SB203580, SB203580(Sulfone), PD169316, SB220025, SKF-86002, SB239063, ML 3163 and a thienyl urea analog (C₁₇H₂ON₂O₃S).

53. Use of an ERK inhibitor and/or a p38 activator for the manufacture of a medicament useful as a contraceptive.

54. The use of claim 53, wherein said ERK inhibitor is selected from the group consisting of a PKC inhibitor, a Ras inhibitor, a dominant negative Ras, a Raf-1 inhibitor, a dominant negative Raf-1, a MEK inhibitor, a dominant negative MEK, a dominant negative ERK and an ERK inhibitor.

55. The use of claim 54, wherein said PKC inhibitor is selected from the group consisting of GF109203X, PKC 19-31, PKC 19-36, staurosporine and G06976.

56. The use of claim 54, wherein said Ras inhibitor is a Ras inhibitory peptide.

57. The use of claim 54, wherein said dominant negative Ras is selected from the group consisting of Ras(S17N) and Ras(S17W).

58. The use of claim 54, wherein said Raf-1 inhibitor is selected from the group consisting of Raf-1 kinase inhibitor I (5-Iodo-3[(3,5-dibromo-4 hydroxyphenyl) methylene]-2 indolinone) and ZM 336372.

59. The use of claim 54, wherein said dominant negative Raf-1 is selected from the group consisting of Raf-1(K375W), Raf(C4B), Raf 301 and Raf(S621A).

60. The use of claim 54, wherein said MEK inhibitor is selected from the group consisting of PD98059, U0126 and U0125.

61. The use of claim 54, wherein said dominant negative MEK is selected from the group consisting of MEK1(K97A), MEK1(K97M), MEK2(K101A) and MEK1/2(KAMEK).

62. The use of claim 54, wherein said dominant negative ERK is selected from the group consisting of ERK1(K71R) and ERK2(K52R).

63. The use of claim 54, wherein said ERK inhibitor is selected from the group consisting of PD98059, PD184352, U0126, ITU, Ste-MEK1₁₃ and MTP_{TAT}-G-MEK1₁₃.

64. The use of claim 53, wherein said p38 activator is selected from the group consisting of p38 activating growth factor, MKK, Rac, Cdc42 and PAK1.

65. The use of claim 64, wherein said p38 activating growth factor is selected from the group consisting of IL-1, IL-1-receptor, TNF, LPS, TRAF6 and TAB1/2.

66. The use of claim 64, wherein said MKK is selected from the group consisting of MKK3, MKK4 and MKK6.

67. The use of claim 64, wherein said Rac is selected from the group consisting of Rac(V12) and Rac(L61).

68. The use of claim 64, wherein said Cdc42 is selected from the group consisting of Cdc42(Q61L) and Cdc42(V12).

69. The use of claim 53, wherein said p38 activator is a condition selected from the group consisting of physical stress, chemical stress and osmotic shock.

70. An article-of-manufacture comprising packaging material and a pharmaceutical composition identified for enhancing fertility being contained within said packaging material, said pharmaceutical composition including, as an active ingredient, an ERK activator and/or a p38 inhibitor and a pharmaceutically acceptable carrier.

71. The article-of-manufacture of claim 70, wherein said ERK activator is selected from the group consisting of Ras, Raf-1, MEK1/2, a PKC activator and UV induction.

72. The article-of-manufacture of claim 71, wherein said Ras is selected from the group consisting of Ras(G12V) and Ras(L61).

73. The article-of-manufacture of claim 71, wherein said Raf-1 is Raf(BXB).

74. The article-of-manufacture of claim 71, wherein said MEK1/2 is ΔN-EEMEK.

75. The article-of-manufacture of claim 71, wherein said PKC activator is selected from the group consisting of TPA, diC8, OAG and arachidonic acid.

76. The article-of-manufacture of claim 70, wherein said p38 inhibitor is selected from the group consisting of Rap, a dominant negative p38, a dominant negative Rac, a dominant negative Cdc42, a dominant negative MKK, a dominant negative Ras, a dominant negative PAK1 and a p38 inhibitor.

77. The article-of-manufacture of claim 76, wherein said dominant negative p38 is p38betaAGF.

78. The article-of-manufacture of claim 76, wherein said dominant negative Rac is Rac(T17N).

79. The article-of-manufacture of claim 76, wherein said dominant negative Cdc42 is Cdc42(T17N).

80. The article-of-manufacture of claim 76, wherein said dominant negative MKK is selected from the group consisting of MKK3(T193A), MKK4(S220A) and a dominant negative MKK6.

81. The article-of-manufacture of claim 76, wherein said dominant negative Ras is selected from the group consisting of Ras(S17N) and Ras(S17W).

82. The article-of-manufacture of claim 76, wherein said dominant negative PAK1 is PAK1(K299R).

83. The article-of-manufacture of claim 76, wherein said p38 inhibitor is selected from the group consisting of 2-(4-Chlorophenyl)-4-(4-fluorophenyl)-5-pyridin-4-yl- 1,2-dihydropyrazol-3-one, SC68376, SB203580(Iodo), SB202190, SB203580, SB203580(Sulfone), PD169316, SB220025, SKF-86002, SB239063, ML 3163 and a thienyl urea analog (C₁₇H₂ON₂O₃S).

84. An article-of-manufacture comprising packaging material and a pharmaceutical composition identified as a contraceptive being contained within said packaging material, said pharmaceutical composition including, as an active ingredient, an ERK inhibitor and/or a p38 activator and a pharmaceutically acceptable carrier.

85. The article-of-manufacture of claim 84, wherein said ERK inhibitor is selected from the group consisting of a PKC inhibitor, a Ras inhibitor, a dominant negative Ras, a Raf-1 inhibitor, a dominant negative Raf-1, a MEK inhibitor, a dominant negative MEK, a dominant negative ERK and an ERK inhibitor.

86. The article-of-manufacture of claim 85, wherein said PKC inhibitor is selected from the group consisting of GF109203X, PKC 19-31, PKC 19-36, staurosporine and GO6976.

87. The article-of-manufacture of claim 85, wherein said Ras inhibitor is a Ras inhibitory peptide.

88. The article-of-manufacture of claim 85, wherein said dominant negative Ras is selected from the group consisting of Ras(S17N) and Ras(S17W).

89. The article-of-manufacture of claim 85, wherein said Raf-l inhibitor is selected from the group consisting of Raf-1 kinase inhibitor I (5-Iodo-3[(3,5-dibromo-4 hydroxyphenyl) methylene]-2 indolinone) and ZM 336372.

90. The article-of-manufacture of claim 85, wherein said dominant negative Raf-1 is selected from the group consisting of Raf-1(K375W), Raf(C4B), Raf 301 and Raf(S621A).

91. The article-of-manufacture of claim 85, wherein said MEK inhibitor is selected from the group consisting of PD98059, U0126 and U0125.

92. The article-of-manufacture of claim 85, wherein said dominant negative MEK is selected from the group consisting of MEK1(K97A), MEK1(K97M), MEK2(K101A) and MEK1/2(KAMEK).

93. The article-of-manufacture of claim 85, wherein said dominant negative ERK is selected from the group consisting of ERK1(K71R) and ERK2(K52R).

94. The article-of-manufacture of claim 85, wherein said ERK inhibitor is selected from the group consisting of PD98059, PD184352, U0126, ITU, Ste-MEK1₁₃ and MTP_{TAT}-G-MEK1₁₃.

95. The article-of-manufacture of claim 84, wherein said p38 activator is selected from the group consisting of p38 activating growth factor, OK, Rac, Cdc42, and PAK1.

96. The article-of-manufacture of claim 95, wherein said p38 activating growth factor is selected from the group consisting of IL-1, IL-1-receptor, TNF, LPS, TRAF6 and TAB1/2.

97. The article-of-manufacture of claim 95, wherein said MKK is selected from the group consisting of MKK3, MKK4 and MKK6.

98. The article-of-manufacture of claim 95, wherein said Rac is selected from the group consisting of Rac(V12) and Rac(L61).

99. The article-of-manufacture of claim 95, wherein said Cdc42 is selected from the group consisting of Cdc42(Q61L) and Cdc42(V12).

100. The article-of-manufacture of claim 84, wherein said p38 activator is a condition selected from the group consisting of physical stress, chemical stress and osmotic shock.

101. A method of enhancing sperm motility comprising:
(a) obtaining a sperm cell sample; and
(b) contacting said sperm cell sample with an ERK activator and/or p38 inhibitor, thereby enhancing sperm motility.

102. The method of claim 101, further comprising:
(c) isolating sperm cells exhibiting enhanced motility from said sperm cell sample.

103. The method of claim 101, wherein said ERK activator is selected from the group consisting of Ras, Raf-1, MEK1/2, a PKC activator and UV induction.

104. The method of claim 103, wherein said Ras is selected from the group consisting of Ras(G12V) and Ras(L61).

105. The method of claim 103, wherein said Raf-1 is Raf(BXB).

106. The method of claim 103, wherein said MEK1/2 is ΔN-EEMEK.

107. The method of claim 103, wherein said PKC activator is selected from the group consisting of TPA, diC8, OAG and arachidonic acid.

108. The method of claim 101, wherein said p38 inhibitor is selected from the group consisting of Rap, a dominant negative p38, a dominant negative Rac, a dominant negative Cdc42, a dominant negative MKK, a dominant negative Ras, a dominant negative PAK1 and a p38 inhibitor.

109. The method of claim 108, wherein said dominant negative p38 is p38betaAGF.

110. The method of claim 108, wherein said dominant negative Rac is Rac(T17N).

111. The method of claim 108, wherein said dominant negative Cdc42 is Cdc42(T17N).

112. The method of claim 108, wherein said dominant negative MKK is selected from the group consisting of MKK3(T193A), MKK4(S220A) and a dominant negative MKK6.

113. The method of claim 108, wherein said dominant negative Ras is selected from the group consisting of Ras(S17N) and Ras(S17W).

114. The method of claim 108, wherein said dominant negative PAK1 is PAK1 (K299R).

115. The method of claim 108, wherein said p38 inhibitor is selected from the group consisting of 2-(4-Chlorophenyl)4-(4-fluorophenyl)-5-pyridin-4-yl- 1,2-dihydropyrazol-3-one, SC68376, SB203580(Iodo), SB202190, SB203580, SB203580(Sulfone), PD169316, SB220025, SKF-86002, SB239063, ML 3163 and a thienyl urea analog (C₁₇H₂ON₂O₃S).

116. A method of determining quality of a semen sample, the method comprising determining p38 activity in sperm cells of the semen sample, said p38 activity being inversely indicative of sperm cell motility, thereby determining the quality of the semen sample.

117. The method of claim 116, wherein determining p38 activity is effected by employing an antiphosphorylated p38 antibody.

118. The method of claim 116, wherein determining p38 activity is effected by a kinase activity assay is an in-gel kinase assay.

119. A kit for determining quality of a semen sample, the kit comprising a container including a reagent suitable for determining p38 activity in sperm cells of the semen sample.

120. The kit of claim 119, wherein said reagent is an antiphosphorylated p38 antibody.

121. The kit of claim 119, further comprising a support for attaching said sperm cells.

122. The kit of claim 119, further comprising reagents suitable for p38 detection.
